(19) United States
(12) Patent Application Publication
(10) Pub. No.: US 2024/0001096 A1
(43) Pub. Date: Jan. 4, 2024

(54) MULTIFUNCTION MICROFLUIDIC OPTRODE

(71) Applicant: UNIVERSITY OF FLORIDA RESEARCH FOUNDATION, INCORPORATED, Gainesville, FL (US)

(72) Inventors: Matthew R. BURNS, Gainesville, FL (US); Yong Kyu YOON, Gainesville, FL (US); Marcelo FEBO, Gainesville, FL (US); Barry SETLOW, Gainesville, FL (US)

(21) Appl. No.: 18/037,364

(22) PCT Filed: Nov. 18, 2021
(86) PCT No.: PCT/US2021/059967

§ 371 Date: May 17, 2023

Related U.S. Application Data
(60) Provisional application No. 63/115,232, filed on Nov. 18, 2020.

(51) Int. Cl. A61M 37/00; A61N 1/05; A61N 5/06; A61N 1/08

(52) U.S. Cl. CPC A61M 37/00; A61N 1/0531; A61N 1/0534; A61N 5/0601; A61N 1/086; A61N 5/0622; A61N 2005/063

(57) ABSTRACT

Disclosed herein is a multifunctional probe that may be inserted into tissue (e.g. brain tissue) that includes an optical waveguide, a microfluidic channel and a plurality of carbon nanofiber electrodes. In another embodiment, the probe includes a shank onto which carbon nanofiber electrodes are disposed. Also disclosed are methods of making disclosed probes.

### BACKGROUND

The discussion of shortcomings and needs existing in the field prior to the present invention is in no way an admission that such shortcomings and needs were recognized by those skilled in the art prior to the present disclosure.

The success of deep brain stimulation (DBS) for diseases as diverse as Parkinson's disease, depression, and obsessive-compulsive disorder suggests that higher cortical functions are orchestrated by deep brain structures. Emerging research has found complex patterns of cortical network activity and disease symptoms driven by the pathology or stimulation of basal ganglia, forebrain, and brainstem nuclei. Despite this success, DBS remains a non-specific electrical stimulation of all cells and tracts in an anatomical target, and the underlying mechanisms of this approach have remained poorly understood. Furthermore, the diverse technical expertise required and high-risk nature of research and development in this field have stifled progress and limited support.

A ‘holy grail’ in neuroscience has been to understand how behavior and disease are ‘encoded’ in local synaptic circuits and in distributed brain networks. Several lines of evidence support a fundamental organizing principle of circuits and networks in the central nervous system (CNS): higher cortical functions like complex movements, memory, emotion, and executive function are orchestrated by deep brain structures. This organizing principle is reflected in symptoms caused by diseases of deep brain nuclei, as well as in the efficacy and side effects observed with a growing number of DBS applications. For example, sequential and progressive involvement of deep brain nuclei precedes later cognitive stages of Alzheimer's and Parkinson's diseases, which involve widespread cortical pathology. In addition, well established DBS protocols in deep brain nuclei ameliorate not only motor symptoms of Parkinson's disease, but can improve depression or obsessive-compulsive disorders in patients, and in some cases improve cognition in animal models of dementia.

Despite the ability of DBS to target deep brain structures and realize beneficial effects across a number of clinical conditions, it remains an empirically-based and non-specific therapy that still fails to address many motor and cognitive symptoms of neurodegenerative diseases while in some cases producing unwanted and unpredictable side effects. Modulating the activity of a discrete node within a complex neural network not only causes nonspecific changes to all cells and tracts at the stimulation site, but to other distributed nodes within functional brain-wide networks. As a result, both the activity and organization of these networks are modulated in what may be node, network, and disease-specific ways that are not easily predicted by traditional theories of cellular and functional anatomy. For example, DBS has been highly successful in treating motor symptoms of Parkinson's disease, but remains a non-specific electrical stimulation of all cells and tracts in an anatomic target. The underlying mechanisms of this approach have remained poorly understood. In addition, DBS fails to address the cognitive and affective symptoms that patients report as contributing most to their quality of life. As a result, a mechanistic understanding of DBS is urgently needed in terms of the structural and functional connectivity at the stimulation site, the specific cell populations stimulated, and its effects on brain-wide functional networks.

### DETAILED DESCRIPTION

### Introduction and Definitions

This disclosure is written to describe the invention to a person having ordinary skill in the art, who will understand that this disclosure is not limited to the specific examples or embodiments described. The examples and embodiments are single instances of the invention which will make a much larger scope apparent to the person having ordinary skill in the art. Unless defined otherwise, all technical and scientific terms used herein have the same meaning as commonly understood by the person having ordinary skill in the art. It is also to be understood that the terminology used herein is for the purpose of describing examples and embodiments only, and is not intended to be limiting, since the scope of the present disclosure will be limited only by the appended claims.

All the features disclosed in this specification (including any accompanying claims, abstract, and drawings) may be replaced by alternative features serving the same, equivalent, or similar purpose, unless expressly stated otherwise. Thus, unless expressly stated otherwise, each feature disclosed is one example only of a generic series of equivalent or similar features. The examples and embodiments described herein are for illustrative purposes only and that various modifications or changes in light thereof will be suggested to the person having ordinary skill in the art and are to be included within the spirit and purview of this application. Many variations and modifications may be made to the embodiments of the disclosure without departing substantially from the spirit and principles of the disclosure. All such modifications and variations are intended to be included herein within the scope of this disclosure. For example, unless otherwise indicated, the present disclosure is not limited to particular materials, reagents, reaction materials, manufacturing processes, or the like, as such can vary. It is also to be understood that the terminology used herein is for purposes of describing particular embodiments only and is not intended to be limiting. It is also possible in the present disclosure that steps can be executed in different sequence where this is logically possible.

All numeric values are herein assumed to be modified by the term “about,” whether or not explicitly indicated. The term “about” generally refers to a range of numbers that one of skill in the art would consider equivalent to the recited value (for example, having the same function or result). In many instances, the term “about” may include numbers that are rounded to the nearest significant figure.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

As used herein, the term “pitch” refers to the average distance between electrode contacts.

As used herein, the term “standard temperature and pressure” generally refers to 25° C. and 1 atmosphere. Standard temperature and pressure may also be referred to as “ambient conditions.” Unless indicated otherwise, parts are by weight, temperature is in ° C., and pressure is at or near atmospheric. The terms “elevated temperatures” or “high-temperatures” generally refer to temperatures of at least 100° C.

Unless otherwise specified, all percentages indicating the amount of a component in a composition represent a percent by weight of the component based on the total weight of the composition. The term “mol percent” or “mole percent” generally refers to the percentage that the moles of a particular component are of the total moles that are in a mixture. The sum of the mole fractions for each component in a solution is equal to 1.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by prior disclosure. Further, the dates of publication provided could be different from the actual publication dates that may need to be independently confirmed.

### General Discussion

Various embodiments utilize nanofabrication, allowing for the development and miniaturization of MRI-compatible implantable neural devices. Various embodiments bring together optogenetic, fluorescent microendoscopy, high-field magnetic resonance imaging modalities. Various embodiments may provide unprecedented specificity in brain stimulation and data collection and may also provide highly detailed electrophysiological, cellular, and structural neural circuit and network data in real time. The all-in-one neural implants, according to various embodiments have the potential to dramatically advance understanding of nervous structure and potential new treatments. Various embodiments provide an integrated, all-in-one MRI-compatible neural implant device with optical, electrical, microfluidic, and wireless functionalities, cutting across scales of neurophysiology with the potential for transformative impact on the field of neuroscience and neurodegeneration.

Developments in the fields of optogenetics, fluorescent microendoscopy, high field magnetic resonance imaging (MRI), and nanofabrication of MR-compatible neural implants offers opportunities to address the limitations of DBS. Various embodiments provide a combination of these modalities into a single device with potential to move the field of neuromodulation forward. For example, various embodiments succeed at combining optogenetics with fMRI or fluorescent microendoscopy with behavior.

A major rationale for the multi-modal approach according to various embodiments to neuromodulation and multi-level network activity measurement arises from the need to address mechanistic questions on multiple physiologic levels simultaneously in the awake behaving animal. Fundamental barriers have been the siloed nature of research techniques at these different scales of physiology—from cells to circuits to networks to behavior—as well as the significant collaborative investment and effort required for the design and optimization of such an integrated device. Bringing these diverse disciplines together towards these fundamental questions remains elusive. To assess changes in activity in cells within a node, their surrounding population of cells, and the broader network comprising anatomically connected regions, requires the type of neuromodulatory technology proposed here. Indeed, integration of these modalities to cut across scales of neurophysiology would have a transformative impact.

Various embodiments provide an integrated all-in-one high-field MRI compatible device with optical, electrical, microfluidic, and wireless functionalities. These all-in-one neural implants have the potential to dramatically advance understanding of the functional organization of the nervous system, and as a result, the pathophysiology of multiple neurologic conditions including Parkinson's disease. Moreover, this technology has the capacity to tailor stimulation parameters to specific pathological states in individual subjects, moving DBS towards precision medicine.

### Wireless Module

Various embodiments relate to a wireless microfluidics optrode for measurement and neuroimaging. According to various embodiments the components and materials used in the optrode are selected for MR compatibility based on tests of their interactions with MR fields. Various embodiments also relate to the programming of software to measure signal components and to deliver electric pulses, microfluidics or lasers to modulate or synch the probe measurements with MRI acquisition using an ex vivo MRI phantom mimicking the chemical environment of brain tissue.

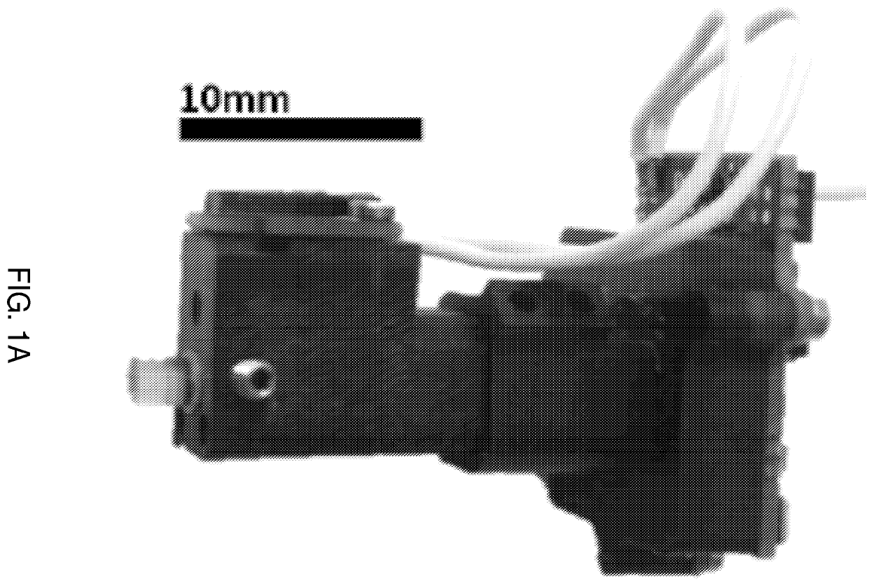

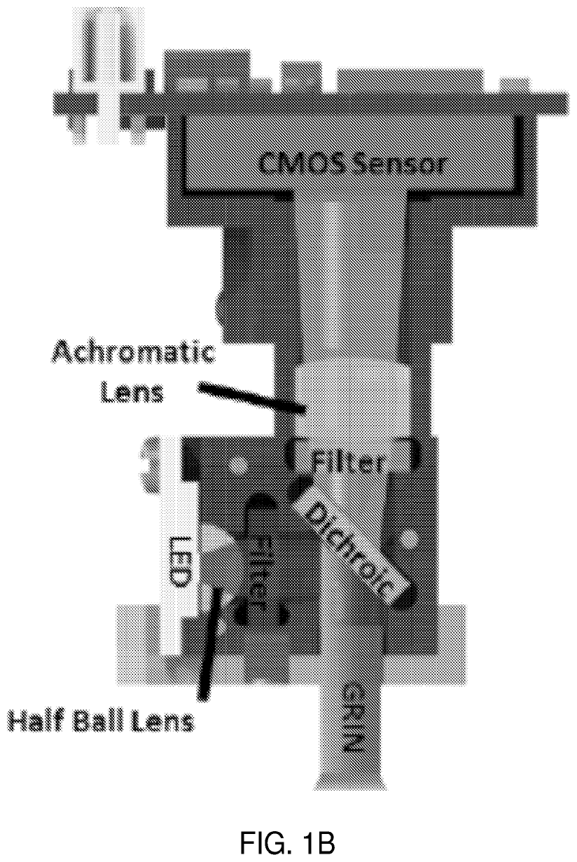

FIG. 1A is an example according to various embodiments illustrating a wireless module with Bluetooth that may be integrated into an optical module with LED lasers (Miniscope). FIG. 1B is an example according to various embodiments illustrating a schematic cross-section view of the wireless module shown in FIG. 1A. FIG. 1C is an example according to various embodiments illustrating an exploded view of the wireless module as shown in FIGS. 1A and 1B.

### Carbon Nanofiber Probe

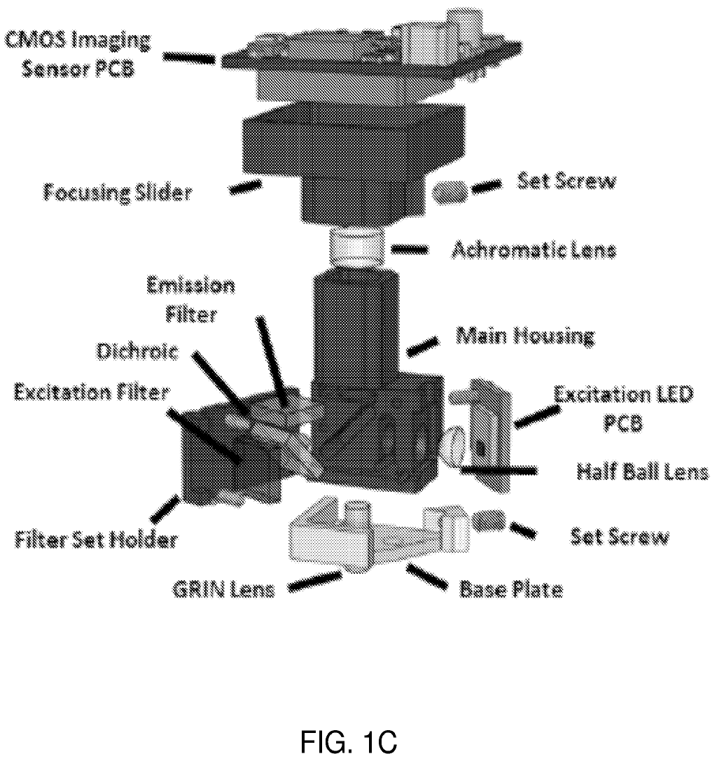

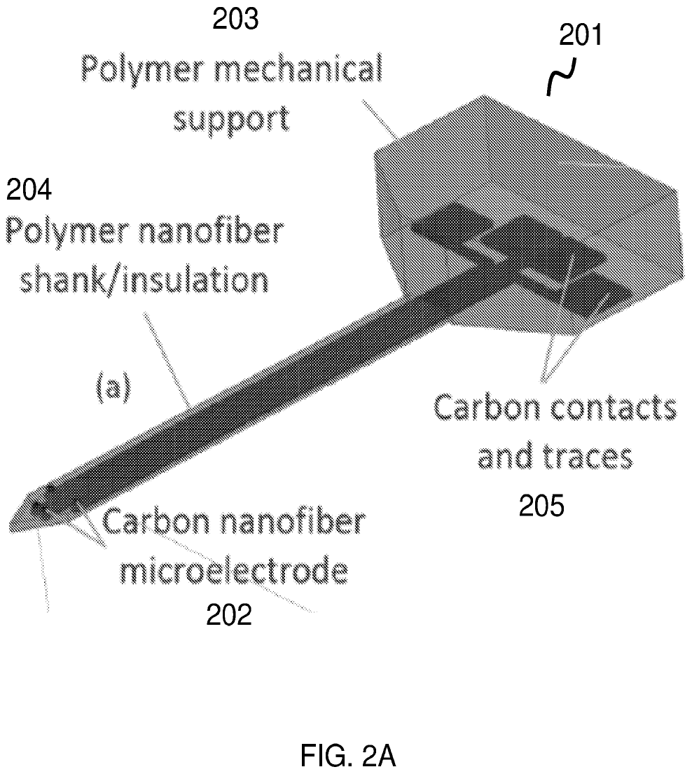

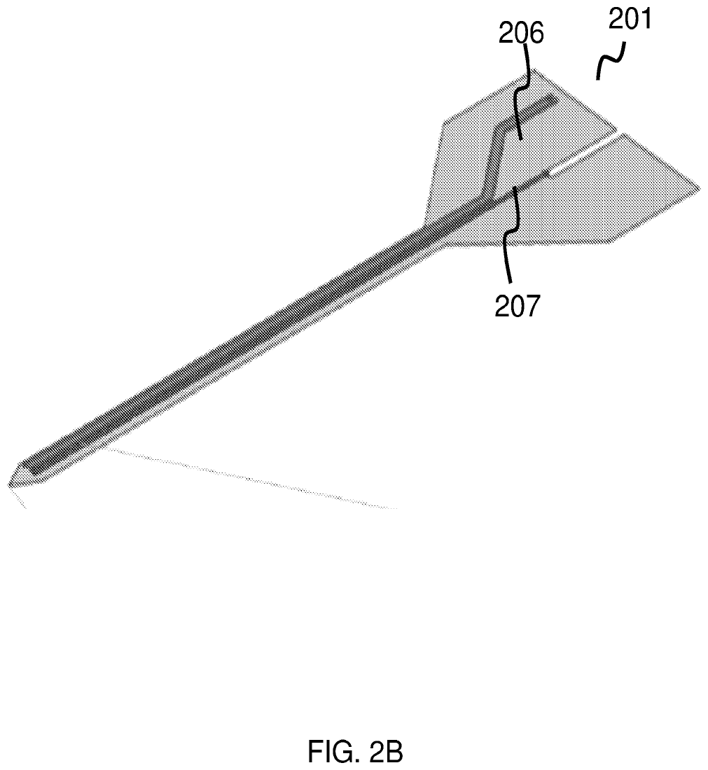

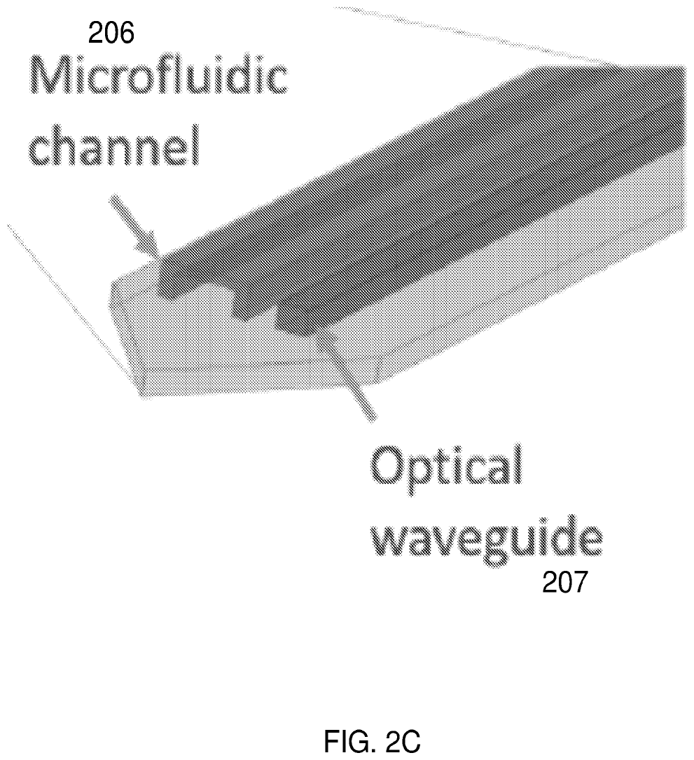

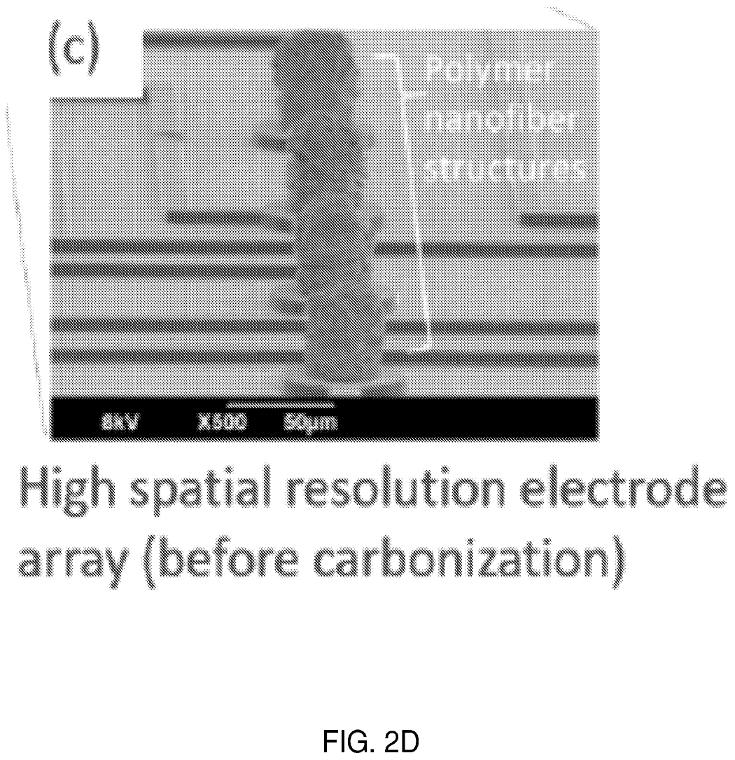

FIG. 2A is an example according to various embodiments illustrating a schematic of the high spatial resolution carbon nanofiber-based neural probe 201 which may be integrated with a microfluidic channel and an optical waveguide. The probe 201 includes 4 carbon nanofiber electrodes 202 with a pitch of 100 μm. The probe 201 may include a polymer mechanical support base 203 and a polymer nanofiber shank 204. The shank 204 typically includes a pointed tip at its distal end to facilitate insertion into tissue (e.g. brain tissue). The shank 204 may insulate and house the carbon nanofiber microelectrodes 202 and may extend to the polymer mechanical support 203. The polymer mechanical support may house and insulate a plurality of carbon contacts and traces 205 electrically coupled to the carbon nanofiber microelectrodes 202. While the polymer mechanical support base 203 is shown as a 3D block, 203 is not drawn to scale and the dimensions may vary such as taking a more planar form.

FIG. 2B is an example according to various embodiments illustrating a possible bottom view of the probe 201 of FIG. 2A, showing the microfluidic channel 206 and the optical waveguide 207 integrated on the backside of the electrode trace. Typically, the microfluidic channel 206 and the optical waveguide 207 will be separated from the electrodes and traces by an insulating layer.

FIG. 2C is an example according to various embodiments illustrating a closeup view of the tip of the probe shown in FIG. 2B, showing the microfluidic channel 206 and the optical waveguide 207 integrated on the backside of the electrode trace.

FIG. 2D is an example according to various embodiments illustrating a scanning electron microscope (SEM) image of an electrode array on a carbon nanofiber neural probe as illustrated schematically in FIG. 1A, before passivation. The electrodes have 30 μm width and 30 μm height with a trace width of 15 μm

### A Process for Producing a Carbon Nanofiber Probe

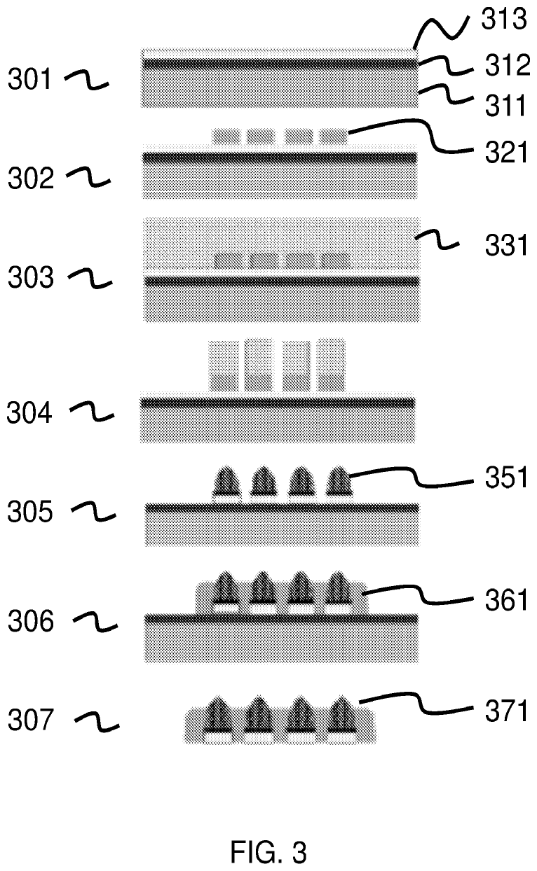

FIG. 3 is an example according to various embodiments illustrating a process for producing a carbon nanofiber electrode with high spatial resolution that may be used in a carbon nanofiber probe. Each step is illustrated by a schematic cross section of the product produced by the step. Generally, the process includes electrospinning a photopatternable epoxy (such as, for example, SU-8, Microchem Inc.), oil emulsion photolithography, and carbonization. The process offers carbon electrodes with nanoscopic morphology, micrometer spatial resolution and an individual nanofiber diameter of 50-200 nm, as shown, for example, in FIG. 2D.

At step 301, a silicon (Si) wafer 311 may be cleaned, a silicon dioxide (SiO₂) layer 312 may be formed onto the silicon wafer 311, and chromium (Cr) layer 313 may be deposited onto the SiO₂ layer 312. The SiO₂ layer may be about 200 nm thick. The SiO₂ layer may be formed by thermal oxidation of the silicon wafer 311. The silicon wafer may be any suitable size, for example, it may have a diameter of about 4 inches. The Cr layer 313 may be about 300 nm thick. The Cr layer 313 may be deposited by sputtering. The Cr layer 313 may be an adhesion promotion layer, meaning that it may help additional layers and components to adhere to the SiO₂ layer 312 of the silicon wafer 311.

At step 302, traces 321 may be patterned on the Cr layer 313. The traces 321 may be about 5 μm thick and may comprise a polymer, such as an epoxy-based negative photoresist (for example, SU-8). The traces 321 may subsequently be converted to carbon traces after pyrolysis. The traces 321 may be patterned using a 3D printer. The line width of the traces 321 may be about 10 μm.

At step 303, a nanofiber layer 331 may be electrospun over the traces 321 and the Cr layer 313. The nanofiber layer 331 may comprise an epoxy-based negative photoresist (such as SU-8). The electrospun nanofibers of the nanofiber layer 331 may have a stack thickness of about 50 μm.

At step 304, some portions of nanofiber layer 331 may be removed by an etching process. For example, portions of the nanofiber layer 331 extending beyond the photoresist 331 may be removed by a photolithographic technique, such as oil immersion photolithography.

At step 305, the Cr layer 313 may be removed and the nanofiber layer 331 may be carbonized via heating to form carbonized nanofibers 351. For example, the entire wafer 311, including the Cr layer 313 and the nanofiber layer 331, and other layers may be pyrolyzed in a nitrogen environment at about 1000° C. for about 1 hour.

At step 306, an epoxy-based negative photoresist (such as SU-8) may be patterned over the carbonized nanofibers 351 to form an insulation layer 361. The photoresist may be about 50 μm thick and may be spin-coated and patterned to open the contact points. For MRI compatibility, glassy carbon that is derived from SU-8 (Microchem Inc.), photosensitive epoxy may be fabricated.

At step 307, the SiO₂ layer 312 may be removed to release the resultant electrodes 371. The probe structure may be separated from the Si wafer by removing the oxide layer selectively using a buffered oxide etchant (BOE).

The resultant carbon nanofiber electrode has many advantages, including, but not limited to: biochemical inertness and stability; nanoscopic surface morphology, which, according to various embodiments, lowers interfacial impedance and enhances neural signal integrity; and electromagnetic compatibility in high magnetic fields and at high frequencies. The material characteristics of the electrospun, nanofiber-derived, carbon structures (for example, the carbonized nanofibers 351) produce almost no effects on MR susceptibility. The material characteristics also minimizes tissue overheating during radio frequency pulsation at 11.1 Tesla. The nanofiber morphology and relatively high electrical resistivity compared with metallic counterparts effectively suppresses Eddy currents, which occur with other metallic electrodes used during MRI. The microfluidic channel and the optical waveguide are realized using the processes of various embodiments. Various embodiments may further include a wireless module with Bluetooth low energy may be integrated with optical module with LED lasers, as shown, for example, in FIGS. 1A-1C. As will be apparent to a person having ordinary skill in the art based on this disclosure, the electromagnetic properties of the carbon structures may be changed by employing different polymer filaments and additives for 3D printing and electrospinning.

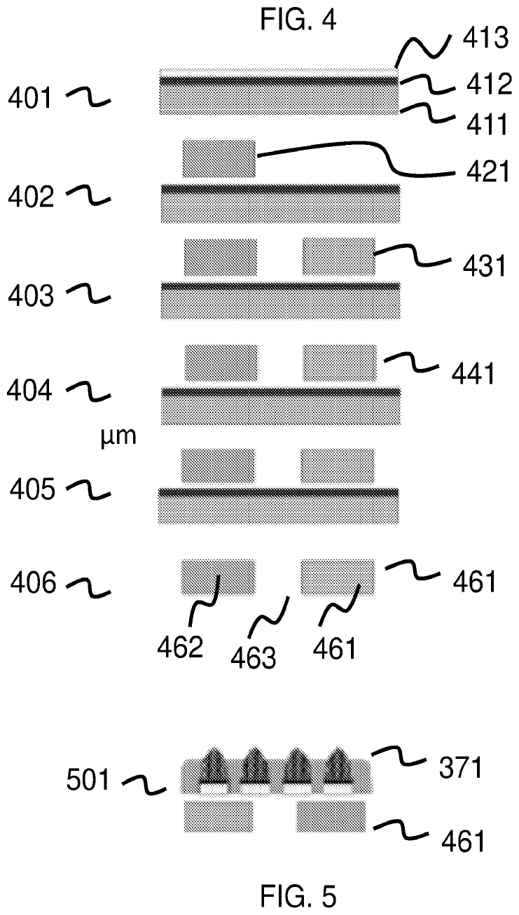

FIG. 4 is an example according to various embodiments illustrating a process for producing a microfluidic channel and optical waveguide. Each step is illustrated by a schematic cross section of the product produced by the step.

At step 401, a silicon (Si) wafer 411 may be cleaned, a silicon dioxide (SiO₂) layer 412 may be deposited onto the silicon wafer 411, and a first polymer layer 413 may be deposited onto the SiO₂ layer 412. The first polymer layer 413 may, for example, be a 5 μm thick layer of a parylene polymer, such as parylene-C.

At step 402, a waveguide 421 may be patterned on the first polymer layer 413. The waveguide 421 may be about 20 μm thick and may comprise an epoxy-based negative photoresist such as SU-8.

At step 403, a block 431 may be patterned on the first polymer layer 413. The block 431 may be about 20 μm thick and may comprise polyethyleneglycol (PEG).

At step 404, a second polymer layer 441 may be deposited over the waveguide 421, the block 431 and the first polymer layer 413. The second polymer layer 441 may be about 5 μm thick and may comprise a parylene polymer, such as Parylene-C. The second polymer layer 441 may have the same or a different composition as the first polymer layer 413.

At step 405, excess portions of the second polymer layer 441 and/or excess portions of the first polymer layer 413 may be removed by reactive-ion etching (RIE). As will be understood by a person having ordinary skill in the art, RIE uses chemically reactive plasma to remove material deposited on wafers. The plasma may be generated under low pressure (vacuum) by an electromagnetic field.

At step 406, the SiO₂ layer 412 may be removed and a component 461 comprising a microfluidic channel 461 and an optical waveguide 462 joined by residual polymer layer 463 may be released. According to various embodiments, the polymer, such as PEG in the microfluidic channel 461 will be dissolved after it is inserted in the brain.

FIG. 5 is an example according to various embodiments illustrating a final step in producing a carbon nanofiber probe 501. The step is illustrated by a schematic cross section of the product produced by the step. The carbon nanofiber probe 501 may be produced by integrating the carbon nanofiber electrode array 371 as produced by the process illustrated in FIG. 3, with the component 461 comprising the microfluidic channel 461 and the optical waveguide 462, illustrated in FIG. 4. The carbon nanofiber electrode array 371 may be integrated with the component 461 by gluing, for example with SU-8.

### Multifunctional Intelligent Neural Probe System

This section describes the physical implementation of carbon nanofiber electrodes, optical and microfluidic components. The advancement of high field fMRI technology has the potential for unprecedented mapping of brain activity based on increased sensitivity to the Blood Oxygen Level Dependent (BOLD) effect. High magnetic fields used in conjunction with strong gradients can lead to very high signal to noise and BOLD contrast. However, ultra-high fields are also more prone than lower fields to magnetic susceptibility artifacts and the generation of heat from eddy currents. The probe proposed here will circumvent these problems and add additional functionalities.

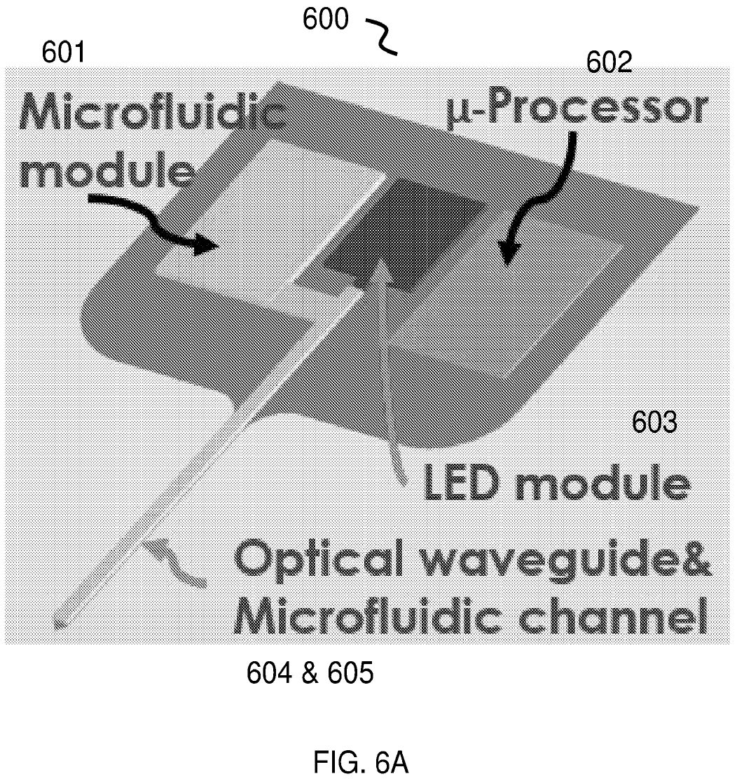

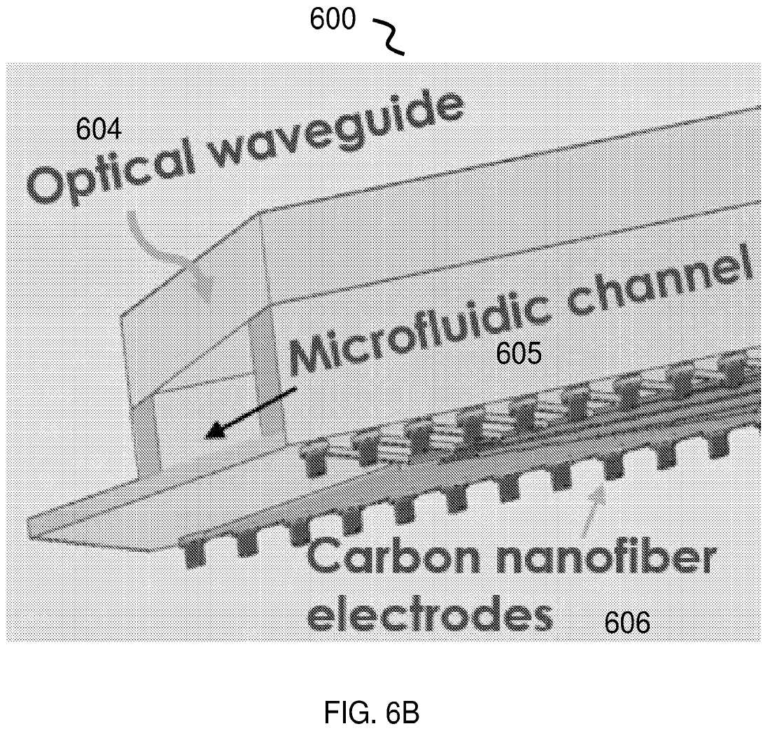

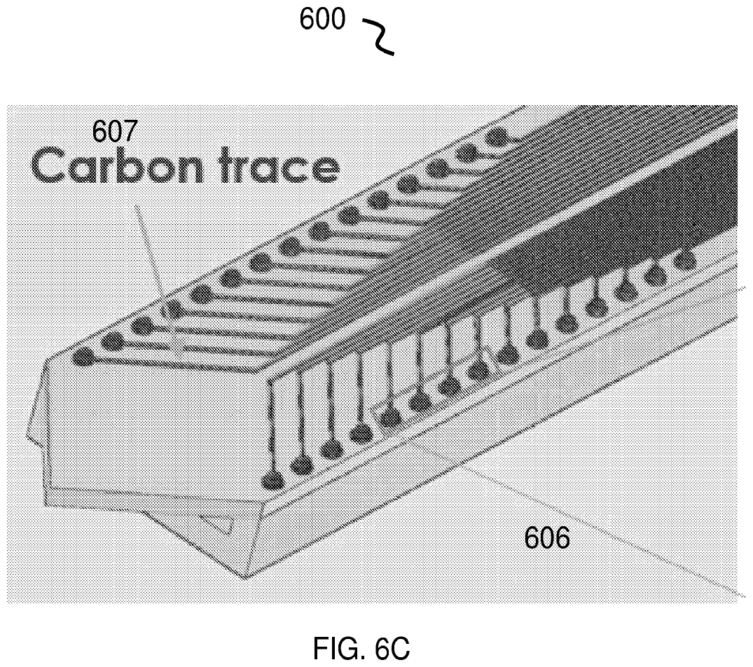

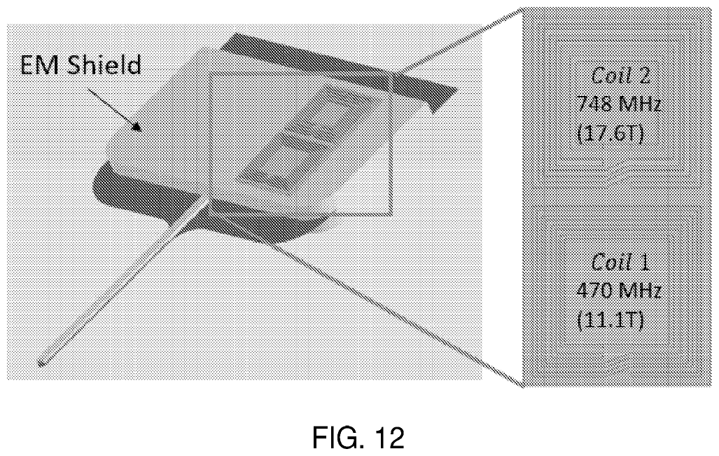

FIG. 6A is an example according to various embodiments illustrating a schematic top view of multifunctional probe 600. The multifunctional probe 600 may be a high spatial resolution carbon nanofiber-based neural probe. The probe 600 may have a microfluidic module 601 fluidically coupled to a microfluidic channel 605. The probe 600 may include an LED (or other light) module 603, optically coupled to an optical waveguide 604. The probe may also include a microprocessor 602 that is electrically connected to the carbon nanofiber microelectrodes 202 and/or carbon contacts and traces 205. Collected electrical signals from the carbon nanofiber microelectrodes 202 will be processed in the microprocessor (can be integrated with a wireless module such as Bluetooth) and delivered to the user computer. The integrated microprocessor will also help generate electrical and optical signals, and control the microfluidic system for delivery or sampling. The microfluidic module 601, LED module 603, and processor 602 are disposed on a mechanical support base 610. The carbon traces are not visible in FIG. 6A as they are disposed on the opposite side of the probe 600. The mechanical support base 610 may have one or more apertures (not shown) such that electrical connections from the carbon nanofiber electrodes 606 can reach the processor 601. FIG. 12 illustrates further components that can be added to the probe 600, which are described further below.

FIG. 6B is an example according to various embodiments illustrating a schematic magnified view of the tip of the multifunctional probe 600 shown in FIG. 6A. The probe 600 includes a microfluidic channel 605 and an optical waveguide 604 on the top. the probe 600 further includes carbon nanofiber electrodes 606 disposed on and underside thereof.

FIG. 6C is an example according to various embodiments illustrating a magnified back backside view of the tip of the multifunctional probe 600 shown in FIGS. 6A and 6B. The probe 600 includes carbon electrodes 606 and traces 607 on the bottom. A 2×15 electrode array is designed with a pitch of 20 μm. According to various embodiments, the neurons of the subthalamic nucleus (STN) and cortex (common targets for clinical DBS) are about 25 to about 40 μm in diameter and the pitch between the electrode contacts is designed to be about 20 μm, enabling multiple parts of the brain to be targeted.

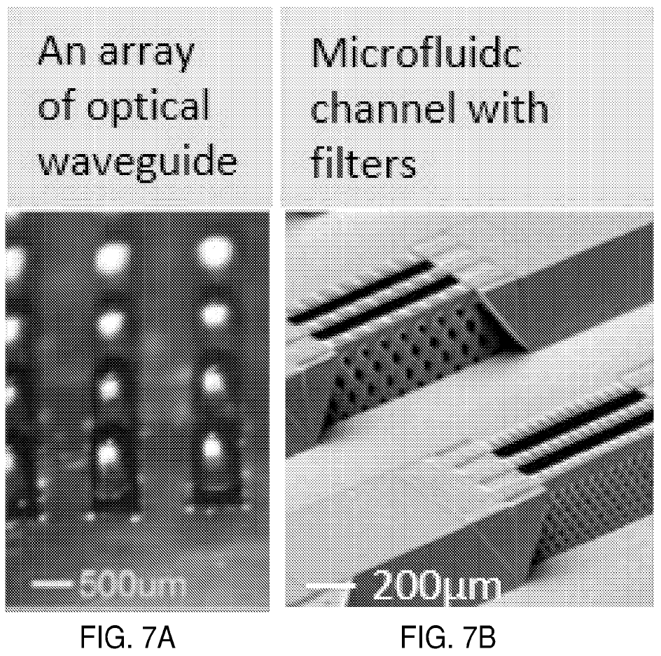

FIG. 7A is an example according to various embodiments illustrating an optical waveguide array, more specifically, a polymeric optical waveguide array with a diameter of 500 μm and a height of 700 μm

FIG. 7B is an example according to various embodiments illustrating an SEM micrograph of microfluidic channel integrated with filters, more specifically, a polymeric microfluidic channel integrated with mesh filters, with a channel width of 500 μm and a channel height of 500 μm.

### Fabrication of a Multifunctional Neural Probe

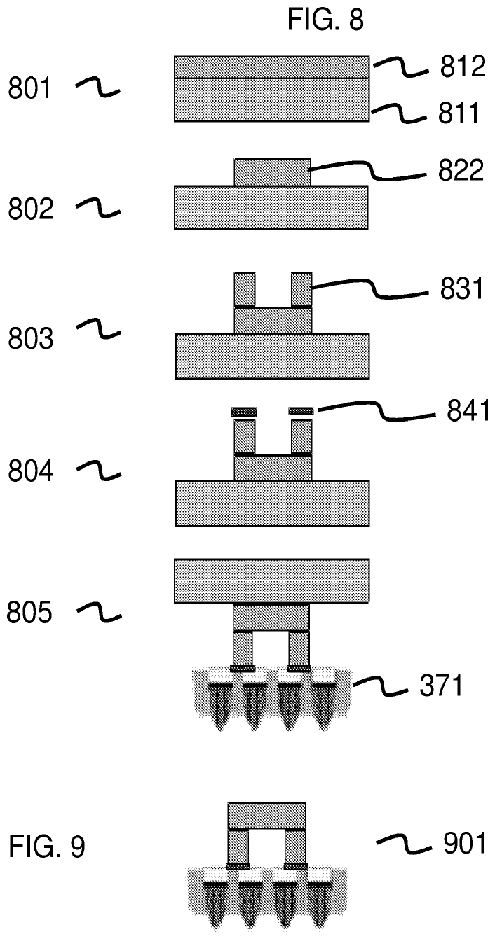

As an overview, a multifunctional neural probe according to various embodiments may be produced by a combination of the process steps illustrated in FIG. 3 and in FIG. 8. For example, a carbon nanofiber electrode 371 may be produced as described with reference to FIG. 3. The carbon nanofiber electrode 371 may then be combined with a microfluidic channel and optical waveguide produced as will be described with reference to FIG. 8. A cross-section of the completed multifunctional neural probe is shown in FIG. 9. Similar structures are also well-illustrated in FIGS. 6A and 6B.

FIG. 8 is an example according to various embodiments illustrating a process for producing a microfluidic channel and optical waveguide and combining the microfluidic channel and optical waveguide with a carbon nanofiber electrode to produce the combined multifunctional neural probe. Each step is illustrated by a schematic cross section of the product produced by the step.

At step 801, a glass wafer 811 may be cleaned and coated with a first polymer layer 812. The first polymer layer may comprise a polymer. The polymer may be, for example, a layer of SU-8. The polymer may be about 100 μm thick. The first polymer layer may be applied via any suitable method, for example, by spin-coating.

At step 802, a waveguide 822 is patterned from the polymer layer 812 using photolithography. In other words, the polymer layer 812 may be photolithographically patterned and the patterned SU-8 structure may be used for an optical waveguide.

At step 803, a second polymer layer may be applied. The second polymer layer may have the same or a different composition as the first polymer layer. The second polymer layer may, for example, comprise SU-8. The second polymer layer may be about 100 μm thick. The second polymer layer may be patterned to form sidewalls 831. The patterning may be accomplished by any suitable method, such as, for example, photolithography. Alternatively, the sidewalls 831 may be deposited by an additive manufacturing process, such as 3D printing. The sidewalls 831 may define boundaries of a microfluidic channel.

At step 804, glue layer 841 may be stamped on one or more top surfaces of the sidewalls 831. The glue layer 841 may be, for example, a layer of polymer, such as SU-8. The glue layer 841 may be about 10 μm thick.

At step 805, a carbon nanofiber electrode 371 may be attached to the sidewalls 831 via the glue layer 841. The carbon nanofiber electrode 371 may be produced according to any suitable process, for example by the process described with reference to FIG. 3.

FIG. 9 is an example according to various embodiments illustrating a cross section of a completed all-in-one neural probe 901 after the silicon wafer 811 has been removed. The design of the completed all-in-one neural probe 901 allows for the waveguide and microfluidics channel to be independent of the CNF electrode design, while they are stacked together, which saves space and reduces fabrication complexity. The optical waveguide may require a cladding layer which has a lower refractive index than the waveguide material. Since the contents within the microfluidic channel are of lower refractive index than the SU-8 used as the waveguide material, it meets the basic requirement for functionality as a waveguide.

### Microfluidic Reservoir and Pump

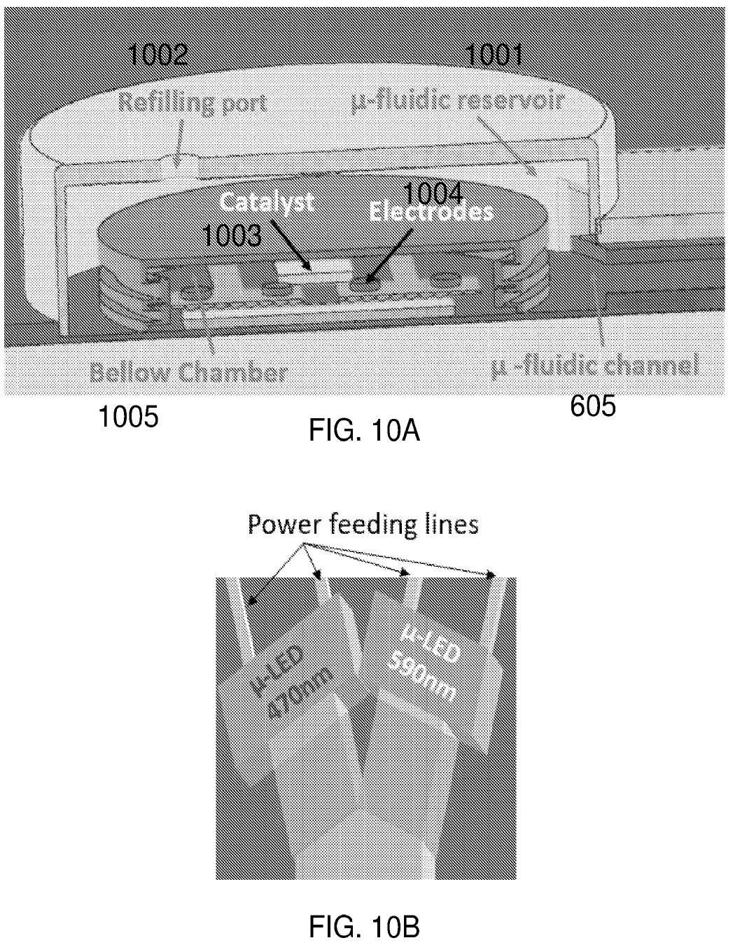

FIG. 10A is an example according to various embodiments illustrating a an example of the microfluidic module 601 shown in FIG. 6A. The microfluidic module 601 is connected with a microfluidic channel 605. As shown in FIG. 6B, an opening is created at the tip of the assembled probe that is fluidically coupled to the microfluidic channel 605. This opening is an outlet. From this opening, fluid or drug may be ejected in the predetermined specific area. The inlet 1007 for the microfluidic channel 605 may be patterned on the mechanical support base 203 where a microfluidic reservoir 1001 and pump 1000 may be integrated. The microfluidic pump 1000 uses a catalytic heater based bellow actuator surrounded by the drug loaded reservoir 1001. When electrical currents are applied to the electrodes 1004 inside the bellow 1005, gas in the bellow chamber is expanded, pumping and squeezing preloaded drug into the channel 605 out the outlet and into the brain (not shown).

FIG. 10B is an example according to various embodiments illustrating micro light emitting diodes (μ-LEDs) as light source to be used in the LED module 603 to provide light to the optical waveguide 604 (see FIG. 6B). Surface mounting device (SMD) high intensity μ-LED (APA1606QBC/D, KingBrightUSA Inc.) may be used as an integrated light source (470 nm and 590 nm) or may be separated. Each will have a dimension of 0.5 mm×1 mm (0204) and the intensity is much greater than 10 mW/cm² appropriate for optogenetic stimulation.

### Integration of Multifunctional Probe and Wireless Module

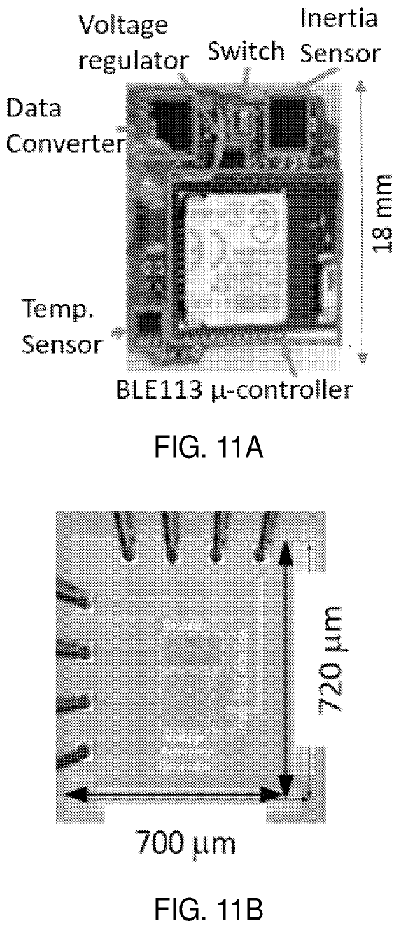

The microprocessor and wireless transceiver (Tx/Rx) module can be powered by a battery or supercapacitor. FIG. 11A is an example according to various embodiments illustrating a μ-controller, Tx/Rx, and sensor module whose footprint is 11 mm×18 mm. A more compact wireless MCU chip (cc2540: 7 mm×7 mm, TI Inc.) integrated with Bluetooth 5.0 may be used. The battery may be charged using a micro-USB connector, but the connector tends to make the system bulky. Therefore, various embodiments may use magnetic resonance based wireless power transfer (WPT), an integrated power regulator circuit, and a supercapacitor.

FIG. 11B is an example according to various embodiments illustrating a compact, ultra-low power consuming voltage regulator circuit whose footprint is only 720 μm×700 μm. A complementary metal oxide semiconductor (CMOS) process (MOSIS ONSemi 0.5 mm) may be used. An MRI-compatible antenna may also be employed.

FIG. 12 is an example according to various embodiments illustrating an integrated probe with the probe handle area covered by electromagnetic (EM) shield, on which two receiver coils are integrated for WPT (coil 1 for 470 MHz in 11.1 T; coil 2 for 748 MHz in 17.6 T). The EM shield layer is made of ferromagnetic and polydimethylsiloxane (PDMS) composites. The coils are designed to harvest RF energy from RF excitation of an MRI system with a resonance frequency of 470 MHz and 748 MHz for 11.1 T and 17.6 T MRI systems, respectively. Each coil has a footprint of 10 mm×10 mm. The coils are connected to the voltage regulation circuits (FIG. 11b) though the shield and then a supercapacitor.

### EXAMPLES

### Introduction

The following examples are put forth to provide those of ordinary skill in the art with a complete disclosure and description of how to perform the methods, how to make, and how to use the compositions and compounds disclosed and claimed herein. Efforts have been made to ensure accuracy with respect to numbers (e.g., amounts, temperature, etc.), but some errors and deviations should be accounted for. The purpose of the following examples is not to limit the scope of the various embodiments, but merely to provide examples illustrating specific embodiments.

### Example 1

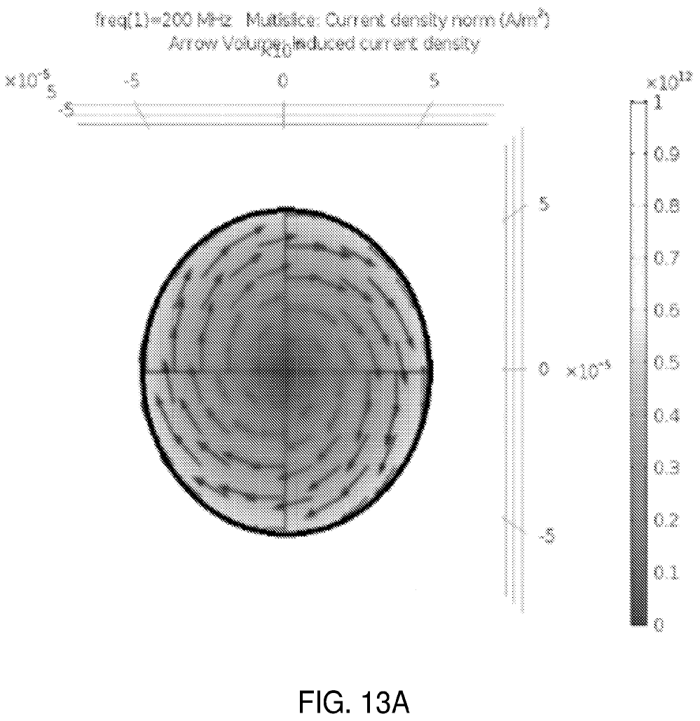

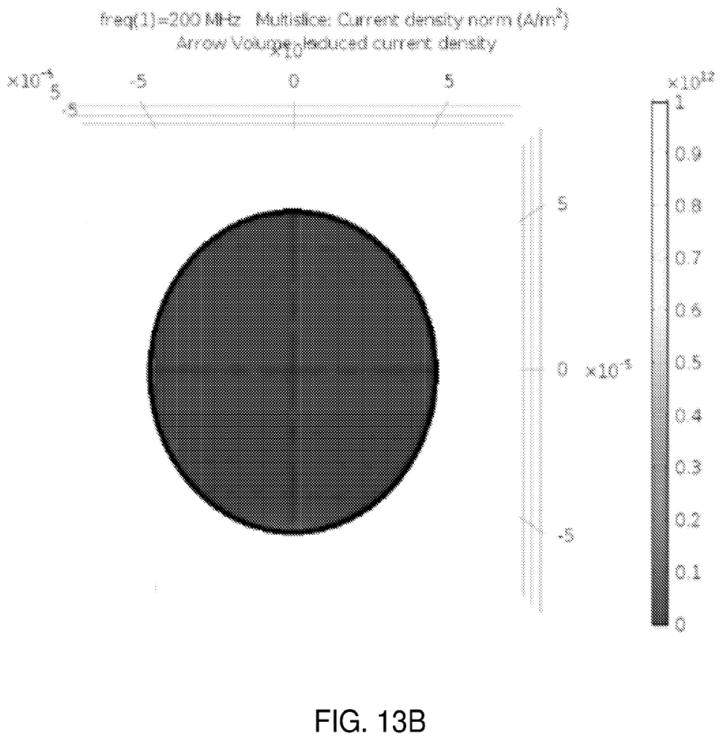

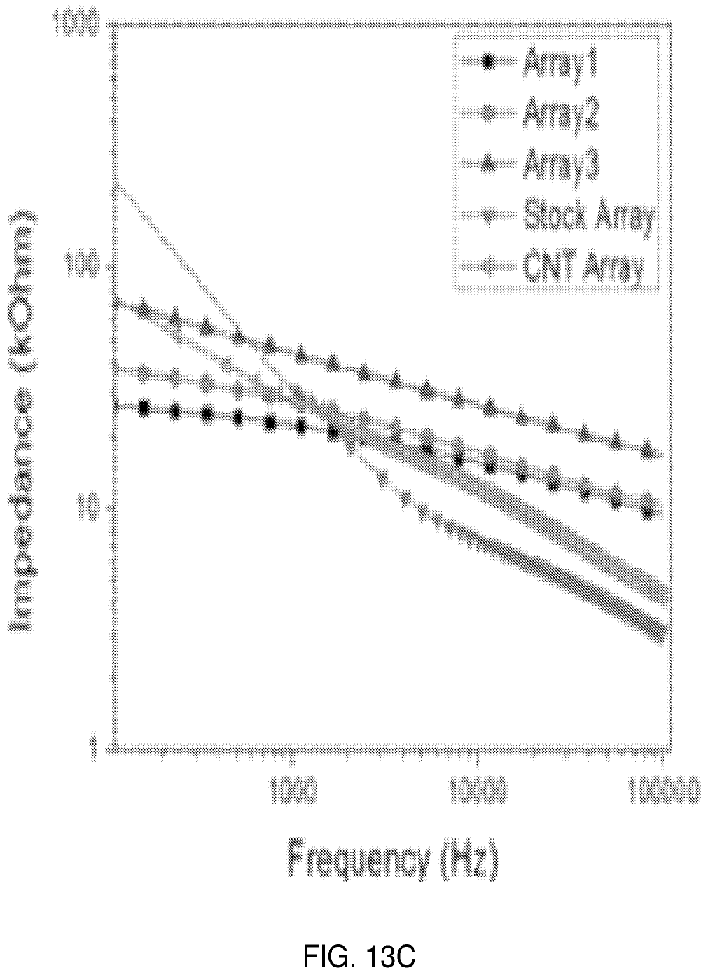

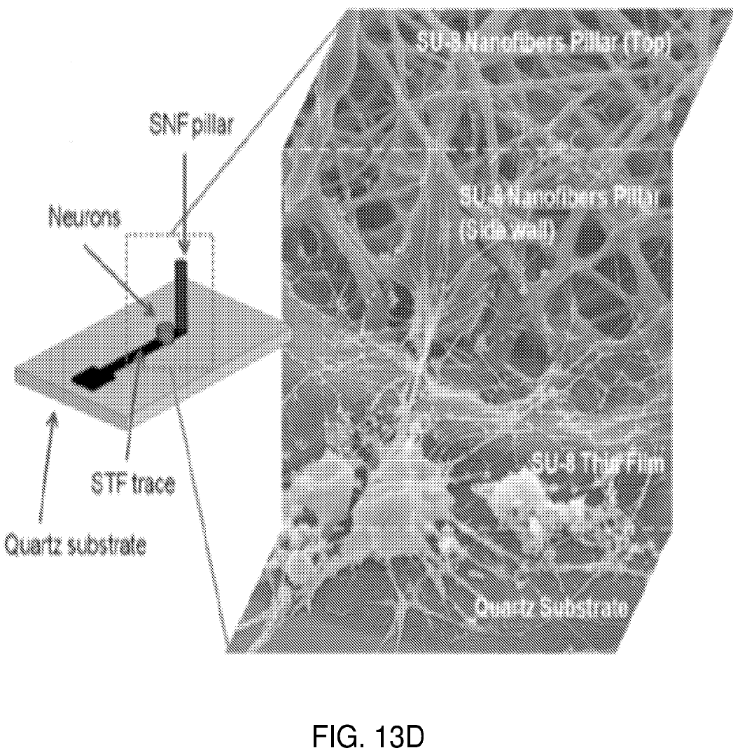

A purpose of this example is to demonstrate that a tungsten (W) electrode shows high Eddy current distribution while the carbon electrode according to various embodiments shows no Eddy current. FIGS. 13A and 13B shows eddy current distribution of a metallic electrode and a carbon electrode using a Multiphysics simulation tool (COMSOL, COMSOL package Inc.). FIG. 13A is an example according to various embodiments illustrating a simulated eddy current of a tungsten electrode. FIG. 13B is an example according to various embodiments illustrating a simulated current of carbon electrode at 470 MHz corresponding to 11.1 T.

FIG. 13C is an example according to various embodiments illustrating the measured impedance of the carbon nanofiber microelectrodes (array 1, array 2, and array 3) have been characterized and compared with those of a carbon nanotube-based electrode and commercially available tungsten (W) electrodes. As used herein, “array 1,” “array 2,” and “array 3” refer to multiple test structures that were made. These test structures are not illustrated. Compared to the impedance of 2-D tungsten electrodes (200 kW), the low impedances of the carbon nanofiber electrodes (at 100 Hz) between 20 kW and 80 kW contribute to better signal integrity (high signal-to-noise).

FIG. 13D is an example according to various embodiments illustrating scanning electron microscope (SEM) view of neuron cultured on nanostructure. The image is colored for clear view: yellow-neuron, blue nanofibers. Also, the in vitro results show enhanced cell survivability on nanofiber electrodes.

### Example 2

A purpose of this example was to demonstrate that since the microfluidic channel and optical waveguides according to various embodiments are made of polymer, they do not interfere with fMRI signals. Furthermore, the microfluidic channel allows direct in situ drug delivery and/or extraction of extracellular matrix media if necessary.

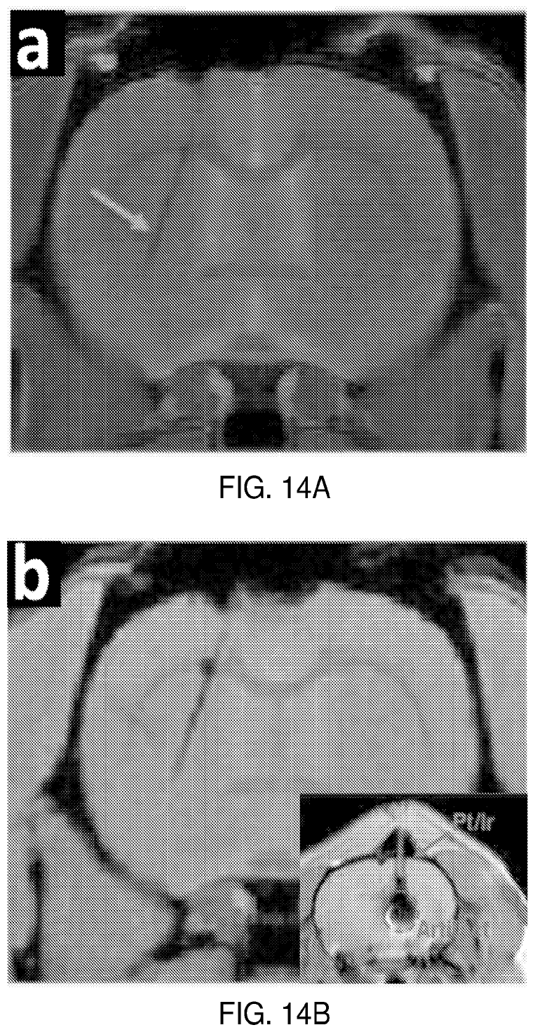

FIGS. 14A and 14B show the recorded artifact-free fMRI images of a fabricated carbon nanofiber electrode neural probe implanted in a rat brain ex-vivo under 4.7 T in spin and gradient echo modes. Inset shows the MRI artifacts caused by a platinum/iridium neural probe at an even lower field strength of 1.5 T. FIG. 14A is an example according to various embodiments illustrating 4.7T fMRI images of the CNF probes implanted in a rat's brain under the spin echo mode. FIG. 14B is an example according to various embodiments illustrating 4.7T fMRI images of the CNF probes implanted in a rat's brain under the gradient echo mode. The inset of FIG. 14B shows the artifact from Pt—Ir probe under a much lower magnetic field (1.5T) MRI.

According to various embodiments, electrodes may be connected by shielded cable to a current stimulator (Grass S48 Stimulator) interfaced with a stimulus isolation unit for constant current delivery. The current source may be controlled by custom programmable LabView software (National Instruments) running on a portable PC Laptop and has +5V TTL inputs/outputs for synchronizing with MRI acquisition. Animals may receive 20 Hz, 150 μA square wave trains, 90 μS pulse duration for 60 minutes. Pulses used for fMRI studies may be brief presentations using boxcar designs (15 sec on, with interleaved 30 sec off epochs).

### Example 3

A purpose of this example is to describe some experimental procedures and goals of future testing.

One aim may be to apply a microfluidics optrode for neuromodulation, measurement and neuroimaging of network activity at rest and in vivo in a wild-type rat model. The phases of this R&D activity will involve surgical placement of the developed probe into several deep brain nuclei known for their role in controlling cortical and thalamic activity (e.g., globus pallidus, nucleus basalis, locus coeruleus) and relevance in Parkinson's disease. Rats will be assessed in high-field MRI to determine baseline relationships between optogenetic/electrical modulation and resting state fMRI brain-wide networks, as well as confirmation of lead localization. Baseline motor and cognitive assessments will also be performed.

Methodologies and Key Personnel: These experiments will employ Long Evans wild type and transgenic rats that express Cre recombinase in dopaminergic, cholinergic, and noradrenergic neurons. Rats (n=6/target) will undergo surgery for bilateral injection of AAV5 vectors (UNC Vector Core) encoding floxed halorhodopsin or channelrhodopsin-mCherry, (or mCherry control) into the above targets, corresponding to well-established coordinates 39, through chronic implantation of the MRI-compatible microfluidics optrode developed according to various embodiments, allowing for both virus delivery and electrical and optical stimulation. Rats are ideal for the current stage of this R&D activity due to the size and weight of the microfluidics optrode. Future work can be scaled in two different directions: the work can be focused to test cell-specificity in Cre-inducible mice or scaled up towards a long term goal of fabricating a device with clinical capabilities. After surgical recovery, both electrical DBS and optogenetic simulation protocols will be evaluated during resting state high field fMRI as described below to assess differential whole brain network stimulation effects, as well as confirming lead placement. In addition, animals will be assessed through a set of standard performance tasks in motor (open field, rotarod), spatial/relational (paired associates learning), attention (5-choice serial reaction time) and executive function (working memory, reversal learning) tasks.

Functional MRI (fMRI): Inventor Febo has 15 years of experience with animal fMRI. There is significant evidence that: (1) resting state fMRI capturing translationally-relevant networks is measurable in rats using established sedation protocols, and, (2) cellular and neurophysiological responses to dopaminergic agents is preserved in distributed networks of anesthetized rats. Thus, we will collect images while rats are sedated using an effective low anesthetic concentration (0.02 mg kg-1 dexmedetomodine [s.c.] and 0.5% isoflurane.

Rats may be prepared for fMRI on an 11.1 T MRI scanner controlled by Bruker Paravision. A transmit/receive surface coil with an opening for cranial implants will be used for signal transmission and detection. Scans will include a high-resolution fast spin echo scan for anatomical overlays and image alignment to atlas space. Two sets of functional scans may be collected, 2 sets of resting state fMRI scans with no stimulation followed by two echo planar images (EPIs) using a block design (six cycles of 30-60 sec off/30-60 sec stimulus on; duration is randomized; 360 repetitions or 12 minutes each scan). A single shot spin EPI scan may be used for acquisition of fMRI images (TR=2 sec; TE=15 ms; 25.6 mm²×20 mm field of view; twelve 0.75 mm interleaved coronal slices (no slice gaps); 64×64 data matrix). In plane spatial resolution is 400 μm² size voxels. Respiratory rates and body temperatures may be monitored throughout setup and acquisition.

Stimulus-induced fMRI and functional connectivity analysis: Previously published processing and analysis strategies may be employed. Anatomical scans may be skull stripped, registered to an annotated rat brain atlas (Ekamsolutions, LLC) and registration matrices applied to functional datasets using FMRIB software library FSL (http://fsl.fmrib.ox.ac.uk/fsl/fslwiki/). Functional images may be corrected for drift and motion. Block design model following the stimulus pattern used in fMRI scanning may be analyzed using first and higher level general linear model analysis with standard tools available in FSL FEAT and with gamma hemodynamic response fit. Resting state scans may be band pass filtered (0.01-0.1 Hz) to remove cardiac and respiratory rhythms. For seed-based analysis, focus may be placed on basal ganglia and cognitive structures; however, connectivity may also be investigated with other anatomical locations. Time series may be extracted from seed regions of interest and used for correlating with the rest of the brain on a voxel-by-voxel basis using Analysis of Functional NeuroImages (AFNI http//afni.nimh.nih.gov/afni/). The resultant Pearson's correlation coefficient maps may be normalized by Fisher's z transformation. Images may be group analyzed using analysis of variance (p<0.05, FDR and/or cluster size corrected). Correlation values may be exported for each region (up to 174) and analyzed by two-factor analysis of variance (DBS vs time as independent variables).

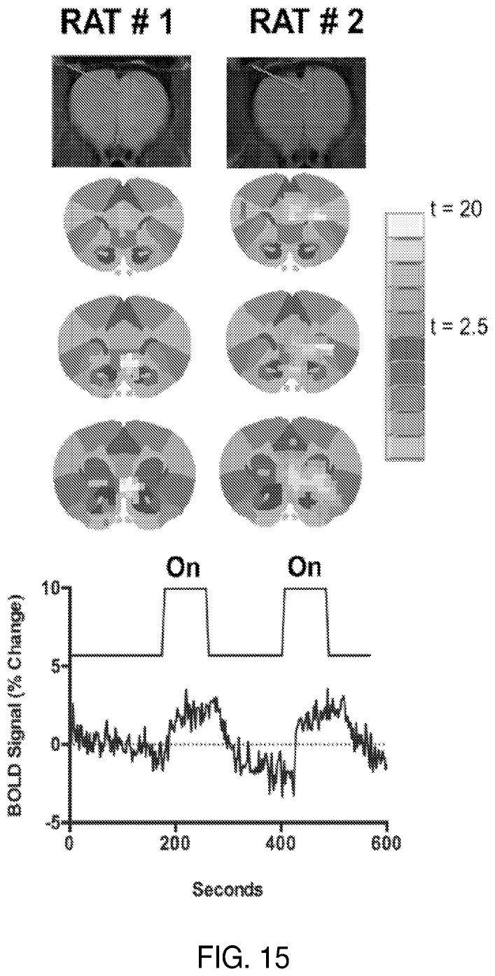

FIG. 15 is an example according to various embodiments illustrating stimulation of PrL prefrontal cortex during fMRI at 4.7 T. Test/re-test study shows activation of ipsilateral NAc. Below is the hemodynamic response using a boxcar design as proposed here for LF DBS of NAc. Febo et al., in progress.

Network analysis: Brain connectivity networks may be analyzed using the Brain Connectivity Toolbox for Matlab. Symmetrical connectivity graphs with a total 11,175 matrix entries may be organized in Matlab. These may be thresholded for each subject to create symmetrical matrices with equal densities (e.g., z values in the top 15% of all possible correlation coefficients). Matrix entries may be normalized by the highest z score, resulting in a range of 0 to 1. Node strength (sum of edge weights), clustering coefficient (the degree to which nodes cluster together in groups), average shortest path length (the potential for communication between pairs of structures), and small worldness (the degree to which rat functional brain networks under study deviate from randomly connected networks) may be calculated for these weighted graphs. The brain networks may be visualized using BrainNet. The 3D networks may be generated with edges that correspond to correlation scores between nodes larger than 0.3.

Laser on/off to electrode on/off in opsin vs. control virus-injected wild-type rat model may be used to establish baseline differences in stimulation parameters and effects. Resting state networks may be assessed using methods previously published. Analyses for fMRI may be as described according to various embodiments, with minor modifications. For behavioral assessments, data may be analyzed using multi-factor ANOVA, with stimulation conditions and brain regions as between-subjects variables, and laser on/off, and electrical stimulation on/off as within-subject variables.

What is claimed is:

1. An magnetic resonance imaging (MRI) compatible device for implantation into a central or peripheral nerve system, the device comprising:
an optical waveguide;
an insulating layer;
a plurality of channel walls disposed between the optical waveguide and the insulating layer to define a microfluidic channel; and
a plurality of carbon nanofiber electrodes distributed across the insulating layer in a first pattern.

2. The device according to claim 1, wherein the first pattern is adapted to facilitate stimulation of at least neurons of a subthalamic nucleus and cortex.

3. The device according to claim 1, wherein the first pattern is adapted to facilitate stimulation of a plurality of brain regions.

4. The device according to claim 1, wherein the optical waveguide is adapted to facilitate optogenetic functionality.

5. The device according to claim 4, further comprising at least one light source adapted to supply light to the optical waveguide.

6. The device according to claim 1, wherein the carbon nanofiber electrodes comprise a polymeric material and a dopant.

7. The device according to claim 6, wherein the polymeric material is selected from the group consisting of epoxy (SU-8), poly lactic acid (PLA), poly urethane, polyimide, polydimethylsiloxane (PDMS), polymethylmetharcrylate (PMMA), polycarbonate, polyethylene terephthalate, and combinations thereof.

8. The device according to claim 6, wherein the dopant is selected from the group consisting of boron, phosphorus, carbon nanotube, graphene, copper nanoparticles, silver nanoparticles, and combinations thereof.

9. The device according to claim 1, wherein the microfluidic channel comprises an inlet and an outlet, and wherein the device further comprises a microfluidic reservoir fluidically coupled to the inlet of the microfluidic channel.

10. The device according to claim 8, further comprising a pump adapted to move fluid from the reservoir to the microfluidic channel.

11. The device according to claim 1, further comprising a microprocessor and wireless transceiver module.

12. The device according to claim 1, wherein each of the plurality of carbon nanofiber electrodes comprises an electrode contact, and
wherein the first pattern comprises a pitch, the average distance between electrode contacts, which adapted to accommodate interaction with separate neurons.

13. The device of claim 12, wherein the pitch for at least one electrode contact is 20 microns or smaller.

14. A method for producing a customized magnetic resonance imaging (MRI) compatible device for implantation into a central or peripheral nerve system, the device comprising an optical waveguide, an insulating layer, a plurality of channel walls disposed between the optical waveguide and the insulating layer to define a microfluidic channel, and a plurality of carbon nanofiber electrodes distributed across the insulating layer in a first pattern,
the method comprising:
defining the first pattern to target one or more specific regions of the central or peripheral nerve system.

15. An magnetic resonance imaging (MRI) compatible device for implantation into a central or peripheral nerve system, the device comprising:
a body comprising an insulating layer; and a plurality of carbon nanofiber electrodes distributed across the insulating layer in a first pattern.

16. The device according to claim 15, wherein the first pattern is adapted to facilitate stimulation of at least neurons of a subthalamic nucleus and cortex.

17. The device according to claims 15 or 16, wherein the first pattern is adapted to facilitate stimulation of a plurality of brain regions.

18. The device according to any of claims 15-17, wherein the carbon nanofiber electrodes comprise a polymeric material and a dopant.

19. The device according to claim 18, wherein the polymeric material is selected from the group consisting of epoxy (SU-8), poly lactic acid (PLA), poly urethane, polyimide, polydimethylsiloxane (PDMS), polymethylmetharcrylate (PMMA), polycarbonate, polyethylene terephthalate, and combinations thereof.

20. The device according to claim 18, wherein the dopant is selected from the group consisting of boron, phosphorus, carbon nanotube, graphene, copper nanoparticles, silver nanoparticles, and combinations thereof.

21. The device according to any of claims 15-21, wherein each of the plurality of carbon nanofiber electrodes comprises an electrode contact, and
wherein the first pattern comprises a pitch, the average distance between electrode contacts, which adapted to accommodate interaction with separate neurons.

22. The device of claim 21, wherein the pitch for at least one electrode contact is 20 microns or smaller.

23. The device according to any of claims 15-22, wherein the device further comprises a support base integrated with the proximal end.

24. The device according to any of claims 15-22 wherein the body comprises a shank.

25. The device of claim 24, wherein the shank comprises a tapered point at the distal end to facilitate insertion into tissue.
